(19) United States
(12) Patent Application Publication
(10) Pub. No.: US 2012/0094363 A1
(43) Pub. Date: Apr. 19, 2012

(54) Method for Producing Non-Putrescible Sludge and Energy and Corresponding Plant

(76) Inventors: Delphine Nawawi-Lansade, Courbevoie (FR); Michel Coeytaux, Angoulins (FR); Stéphane Deleris, Villepreux (FR)

(21) Appl. No.: 13/255,021

(22) PCT Filed: Mar. 8, 2010
(86) PCT No.: PCT/EP2010/052900

§ 371 Date: Dec. 20, 2011

(30) Foreign Application Priority Data
Mar. 6, 2009 (FR) 0951443

(51) Int. Cl. C02F 3/34; C12M 1/00

(52) U.S. Cl. 435/262; 435/290.1

(57) ABSTRACT

The invention relates to a method for producing non-putrescible sludge and energy, wherein said method includes the following steps: (i) producing digested sludge by means of primary sludge digestion; (ii) producing a first aqueous effluent and digested sludge, which are at least partially dehydrated by a first liquid-solid separation of the digested sludge produced in step (i); (iii) producing digested sludge at least partially dehydrated and hydrolysed by thermal hydrolysis of the at least partially dehydrated digested sludge produced in step (ii); (iv) digesting the at least partially dehydrated and hydrolysed digested sludge produced in step (iii); wherein said method further includes a step of recovering the biogases formed during said digestion and said primary digestion, and a step of producing energy from said biogas, including a first sub-step of producing the energy required for implementing said thermal hydrolysis and a sub-step of producing excess energy, the entirety of the biogas being used for producing electricity.

### 1. FIELD OF THE INVENTION

The field of the invention is that of the treatment of organic wastes, especially those produced during water treatment.

More specifically, the invention pertains to a process for treating sludge from the treatment of municipal and industrial water, especially with a view to producing energy, for example electricity.

### 2. PRIOR ART

Municipal or industrial wastewater contains a certain proportion of soluble and particulate organic pollution.

The particulate portion of the pollution can be partly removed by simple decantation. The decantation of the water is accompanied by the formation of sludge, known as “primary sludge” consisting of a mix of particles and water that constitutes organic waste.

The soluble organic portion of the pollution, at least a major part of it, can be treated by the application of biological treatment processes.

The biological treatment of water consists in placing the water to be treated in contact with microorganisms which, in order to ensure their own growth, consume the organic pollution dissolved in this water.

The biological treatment of water is accompanied by the formation of sludge, known as “biological sludge” or “secondary sludge”, constituting organic wastes.

The mix of primary sludges and secondary sludges constitutes “mixed sludges”. To treat these mixed sludges in order to break them down and make them non-putrescible and inoffensive, various techniques have been proposed.

The digestion, or methanation, of organic wastes is a natural process for breaking down organic wastes biologically by subjecting them to anaerobic fermentation.

Digestion is particularly efficient in that it leads to the combined production of:
gas (biogas) convertible into energy or energies;
digestate which can be used for example as a fertilizing agent (a digestate is a residue of the digestion of an organic compound) and
a relatively restricted quantity of low-biodegradable or non-biodegradable solubilized compounds.

However, the digestates thus obtained contain a unreadily biodegradable fraction, i.e. difficult to degrade biologically.

In order to overcome this drawback, the technique has been developed for implementing a thermal hydrolysis of sludges prior to the implementation of a digestion.

This technique is particularly advantageous inasmuch as thermal hydrolysis enables the degradation, at least to a great extent, of the unreadily fermentable (i.e. difficult to ferment) fraction of the sludge.

### 3. DRAWBACKS OF THE PRIOR ART

However, although thermal hydrolysis provides for an appreciable improvement in the elimination of the unreadily fermentable fraction of the sludge, the trade-off is that it entails a greater production of low-biodegradable or non-biodegradable soluble compounds (with high COD or chemical oxygen demand) than is the case in classic digestion. This dictates limits on the quantity of sludges at entry into the digester in order to ensure efficient digestion.

Besides, the conditions needed for obtaining efficient thermal hydrolysis entail high energy consumption.

The energy consumption is such that half of the biogas coming from the digestion is used to feed a classic boiler in order to produce the steam needed for hydrolysis. The rest of the biogas feeds a co-generation motor connected to an alternator in order to produce electricity. It may also for example be used to directly heat buildings.

Thus, this technique, which of course enables the production of digestates with a relatively small concentration of unreadily fermentable fractions gives rise to the following:

it leads to the production of low-biodegradable or non-biodegradable soluble compounds;
it necessitates the oversizing of the digester in order to ensure efficient digestion;
it requires the consumption of a major part of the biogas to directly produce the steam needed for hydrolysis, and therefore enables the production of only a small quantity of surplus energy, for example in the form of electricity, heat etc which can be used for purposes other than implementing the sludge-treatment process in itself.

### 4. GOALS OF THE INVENTION

It is an aim of the invention in particular to overcome these drawbacks of the prior art.

More specifically, it is a goal of the invention, in at least one embodiment, to provide a technique of this kind that requires low energy consumption.

In particular, the invention is aimed at procuring, in at least one embodiment, a technique of this kind whose implementation leads to restricting the consumption of biogas needed to achieve conditions of hydrolysis, and to increasing the share of the biogas that can be used to produce excess energy that can be used for purposes other than that of the implementation of the sludge treatment process.

It is another goal of the invention, in at least one embodiment, to provide a technique for the treatment of sludge coming from the treatment of water, that enables the unreadily fermentable fraction to be eliminated from it, at least to a major extent.

In particular, it is a goal of the invention to implement a technique of this kind, in at least one embodiment of the invention, enabling the production of wastes containing a unreadily fermentable residual fraction that is reduced as compared with the prior art techniques.

The invention, in at least one embodiment of the invention, is also aimed at limiting the production of low-biodegradable or non-biodegradable soluble compounds.

It is yet another goal of the invention, in at least one embodiment of the invention, to provide a technique of this kind for the treatment of large quantities of sludges.

The invention is also aimed, in at least one embodiment of the invention, at providing a technique of this kind that is reliable, simple to implement and relatively economical.

### 5. SUMMARY OF THE INVENTION

These goals, as well as others that shall appear here below are achieved by means of a method for producing essentially non-putrescible sludge and energy, said method comprising the following steps:
(i) obtaining digested sludges by primary sludge digestion;
(ii) obtaining a first aqueous effluent and digested sludges at least partly dehydrated by a first liquid-solid separation of the digested sludges obtained at the step (i);
(iii) obtaining digested sludges at least partially dehydrated and hydrolyzed by thermal hydrolysis of the at least partially dehydrated, digested sludges obtained at the step (ii);
(iv) digestion of the at least partially dehydrated and hydrolyzed sludges obtained at the step (iii);

said process further comprising:
a step for recovering biogases formed during said digestion and said primary digestion and
a step for producing energy from said biogas comprising a sub-step for producing energy needed to implement said thermal hydrolysis and a sub-step for producing surplus energy, the entirety of said biogas being used to produce electricity.

It will be noted that, as understood in the present invention, the term “thermal hydrolysis” shall be understood to refer to a mode of hydrolysis that is expressly non-biological.

Thus, the invention relies on an original approach combining the successive implementation of a first digestion, a (non-biological) thermal hydrolysis and a second digestion of sludge.

The first digestion (or primary digestion) is used to degrade the readily fermentable fraction of the sludge, at least in major part, and to produce a unreadily fermentable digestate.

The implementation of the separation step enables the discharge of an effluent containing the low-biodegradable or non-biodegradable organic matter produced during the digestion. The quantity of low-biodegradable or non-biodegradable organic matter at entry into the hydrolysis step is thus reduced. This ultimately diminishes the quantity of low-biodegradable or non-biodegradable organic matter produced during hydrolysis. In addition, it reduces the size of the equipment placed downstream and reduces the energy consumption needed to carry out the thermal hydrolysis.

The thermal hydrolysis is implemented only to treat the unreadily fermentable fraction of the sludge. The result of this is that the energy needed to implement thermal hydrolysis according to the invention is lower than that needed for thermal hydrolysis in the prior art. Indeed, in the prior art, thermal hydrolysis is carried out to treat all the sludges, i.e. both their fermentable part and their unreadily fermentable part. This calls for a greater input of energy.

Thermal hydrolysis enables the degradation of the unreadily fermentable digestate into an readily fermentable hydrolyzed digestate.

These fermentable sludges are then digested during the second digestion which leads to the production of a digestate free, at least in major part, of a fermentable fraction, the digestate however containing a very unreadily fermentable portion which is also called a refractory or hard fraction.

Furthermore, since the thermal hydrolysis is done only on the unreadily fermentable fraction of the sludges, its implementation leads to the production of a smaller quantity of low-biodegradable or non-biodegradable soluble compounds than in the prior art.

A process according to the invention enables the production of a major quantity of biogas. In addition, the energy needed to carry out the hydrolysis is relatively small since it is done only on the unreadily fermentable part of the sludges. The use of the technique of the invention therefore enables the production firstly of the energy needed to achieve especially the conditions of pressure and temperature for hydrolysis and secondly of a substantial part of surplus energy that can be used for purposes other than those of implementing the process of treating sludges in itself (electricity for example to power a plant or else to be resold to a power supply company, heat (heated fluid (liquid or gas)) to heat buildings etc.

According to one advantageous characteristic, a process according to the invention comprises a step for reconverting said biogases, said reconverting step comprising a step for feeding a co-generation system with biogas in order to produce the energy needed to implement said step of hydrolysis and in order to produce surplus energy.

The feeding of biogas to a co-generation system therefore makes it possible firstly to produce the energy needed to attain especially the conditions of pressure and temperature for hydrolysis and secondly to produce a major part of surplus energy which can be used for purposes other than those of implementing the sludge-treatment process in itself (electricity for example to power a plant or else to be resold to a power supply company, heat (heated fluid (liquid or gas)) to heat buildings etc.

According to another advantageous characteristic, said reconversion step comprises a step for feeding biogas to a motor linked to electricity production means and a step for recovering the heat released by said motor in order to attain the conditions of temperature and pressure for said hydrolysis step.

The entirety of the biogas formed during digestion feeds the cogeneration motor which is connected to electricity production means such as an alternator. The recovery of the heat released by the motor (for example recovered from the exhaust gases and/or oil and/or cooling fluid) enables the production of all the thermal fluid needed to carry out thermal hydrolysis. Thus, according to the invention, the entirely of the biogas is used to produce electricity unlike in prior art techniques where at least 50% of the biogas is used to produce electricity by the implementing of a co-generation motor, the remaining gas feeding a classic boiler to produce, in major part, the thermal fluid used to obtain conditions of pressure and temperature needed to carry out hydrolysis.

Preferably, a process according to the invention comprises a step for obtaining a second aqueous effluent and treated sludges by a second liquid-solid separation of the sludges obtained at said step (iv).

The implementation of this separation step enables the discharge of an effluent containing the low-biodegradable or non-biodegradable organic matter produced during digestion and dehydrated, digested sludges free of readily fermentable organic matter.

Advantageously, said thermal hydrolysis is performed at a pressure of 1 to 20 bars, at a temperature of 50° C. to 200° C., and preferably of 120° C. to 180° C., for a duration of 20 to 120 minutes.

Conditions of thermal hydrolysis chosen in these intervals enable the efficient reduction of the unreadily fermentable portion of the sludges.

According to one valuable variant, said thermal hydrolysis is preferably carried out at a pressure equal to the saturation vapour pressure, at a temperature of 165° C., for a duration of 30 minutes.

These particular conditions of thermal hydrolysis enable optimal reduction of the unreadily fermentable portion of the sludges.

According to one advantageous characteristic, said primary digestion and/or said digestion are of a mesophilic anaerobic type.

In this case, the digestion operation or operations are carried out at a temperature ranging from 32 to 38° C. from 5 to 15 days.

According to another advantageous characteristic, said primary digestion and/or said digestion are of a thermophilic anaerobic type.

In this case, the digestion operation or operations are carried out at a temperature ranging from 52 to 58° C. for 5 to 15 days.

The concentration of matter in suspension at entry into the primary digestion operation ranges from 25 to 65 grams of MIS (Matter In Suspension)/1 of sludges.

The concentration of matter in suspension at entry into the digestion operation ranges from 100 to 150 grams of MIS/1 of sludges.

According to one advantageous characteristic, said step of liquid-solid separation is preceded by a step for defibrating said sludges after primary digestion.

In one variant, the defibrating step can be performed before the primary digestion step.

The defibration makes it possible especially to:
enable sludge treatment considered by those skilled in the art to be impossible with prior art techniques;
reduce the size of the digester placed upstream or downstream,
increase the residence time of the other organic fractions of the sludge.

The invention also covers a sludge-treatment plant to implement a method according to the invention, said plant comprising means of thermal hydrolysis having an inlet and an outlet and means for digesting said sludges.

According to the invention, said digestion means communicate with means for bringing in sludges and said inlet and said outlet of said hydrolysis means communicate with said digestion means, said plant also comprising first liquid-solid separation means positioned at the outlet of said digestion means and means for recovering biogas coming from said digestion means.

Again according to the invention, said digestion means are connected to biogas recovery means which include a collector linked to means for producing steam and electricity comprising a co-generation motor linked to an alternator producing electricity, an exhaust line of which leads into the inlet of an air-water heat exchanger producing steam and a piping used to convey steam to said thermal hydrolysis means.

Such a plant enables the implementing of a process according to the invention, the principle of which relies on the combined implementation of a first digestion, a thermal hydrolysis and a second digestion of the sludges.

The implementation of separation means enables the discharge of an effluent containing low-biodegradable or non-biodegradable organic matter produced during the digestion. The quantity of low-biodegradable or non-biodegradable soluble organic matter at entry into the hydrolysis step is thus reduced, thus ultimately tending to reduce the quantity of low-biodegradable or non-biodegradable organic matter produced during this hydrolysis.

A plant according to the invention includes a system of co-generation, said biogas recovery means communicating with said co-generation system.

The feeding of biogas to a co-generation system enables the production of the energy needed to attain especially the conditions of pressure and temperature for hydrolysis and to produce a substantial portion of surplus energy (for example in the form of electricity and/or heat (hot fluid (air and/or water)) which can be used for purposes other than those of implementing the sludge-treatment process per se.

Preferably, said co-generation system includes a co-generation motor, said biogas recovery means leading into said motor, said co-generation motor being linked to electricity production means and having means to transfer the heat released by said motor into water in order to produce steam.

The entirety of the biogas formed during the digestion operations feeds the co-generation motor which is linked to electricity production means such as an alternator. The recovery of the heat released by the motor (for example recovered from the exhaust gases and/or oil and/or cooling liquid) enables the production of all the thermal fluid (for example steam) needed to perform thermal hydrolysis. Thus, according to the invention, the entirety of the biogas is used to produce electricity unlike in the prior art technique in which at least 50% of the biogas is used to produce electricity by the implementation of a co-generation motor, the remaining biogas feeding a classic boiler to produce, in major part, the thermal fluid used to obtain the conditions of pressure and temperature needed to perform hydrolysis.

According to an advantageous characteristic, said digestion means comprise a digester having at least one inlet and one outlet, said outlet communicating with said inlet of said hydrolysis means and said inlet communicating with said outlet of said hydrolysis means.

According to another advantageous characteristic, said digestion means comprise a primary digester and a secondary digester, said primary and secondary digesters each having an inlet and an outlet, the inlet of said primary digester communicating with said means for bringing in sludges, the outlet of said primary digester communicating with the inlet of said hydrolysis means, the inlet of said secondary digester communicating with the outlet of said hydrolysis means.

Preferably, said first liquid-solid separation means are configured to make it possible to attain a dryness level equal to or greater than 12%.

Advantageously, a plant according to the invention comprises second liquid-solid separation means positioned at the outlet of said secondary digester.

The implementation of these second separation means enables the discharge of an effluent containing low-biodegradable or non-biodegradable soluble organic matter produced during the digestion and dehydrated, digested sludges free of fermentable organic matter.

According to a preferred characteristic, a plant according to the invention includes defibration means positioned between said digester and said separation means or between said primary digester and said first separation means.

In one variant, the defibration means are placed upstream to said digester or primary digester.

The implementation of such defibration means makes it possible especially to:
treat sludges considered by those skilled in the art to be impossible to treat by implementing the prior art technique;
reduce the size of the digester placed upstream or downstream,
increase the residence time of the other organic fractions of the sludge.

Advantageously, said co-generation motor has an exhaust line leading into an air-water heat exchanger with a steam discharge outlet connected to said thermal hydrolysis means. This implementation enables the simple and efficient production of the steam needed to carry out the thermal analysis.

### 6. LIST OF FIGURES

Other features and advantages of the invention shall appear more clearly from the following description of preferred embodiments, given by way of simple illustratory and non-exhaustive examples and from the appended drawings, of which:

FIG. 1 is a drawing of a first embodiment of a plant according to the invention;

FIG. 2 is a drawing of a second embodiment of a plant according to the invention;

FIGS. 3 and 4 are graphs representing the sugar content in sludges respectively before and after the first digestion.

### 7. DESCRIPTION OF AN EMBODIMENT OF THE INVENTION

### 7.1. Reminder of the Principle of the Invention

The invention pertains to a process of sludge treatment. As understood in the invention, the term “sludges” includes primary sludges, secondary sludges and especially mixed sludges.

The general principle of the invention relies on the combined implementation of a first digestion, a thermal hydrolysis and a second digestion of the sludges.

The first digestion enables the degradation, at least in major part, of the readily fermentable fraction of the sludge and the production of a unreadily fermentable digestate.

The thermal hydrolysis is then implemented only to treat the unreadily fermentable fraction of the sludges.

On the contrary, in the prior art, thermal hydrolysis is conducted in order to treat all the sludges, i.e. both the fermentable part and the unreadily fermentable part.

The result of this is that the energy needed to implement the thermal hydrolysis according to the invention is smaller than that needed to carry out the thermal hydrolysis according to the prior art.

Thermal hydrolysis enables the degradation of the digestate coming from the primary digester which is constituted by the unreadily fermentable fraction of the sludges and the production of a hydrolyzed digestate consisting of readily fermentable sludges.

The second digestion then enables the digestion of these fermentable sludges and the production of a digestate which is free, at least in major part, of any fermentable fraction and contains only a small refractory non-fermentable portion.

### 7.2. Example of a First Embodiment of a Plant According to the Invention

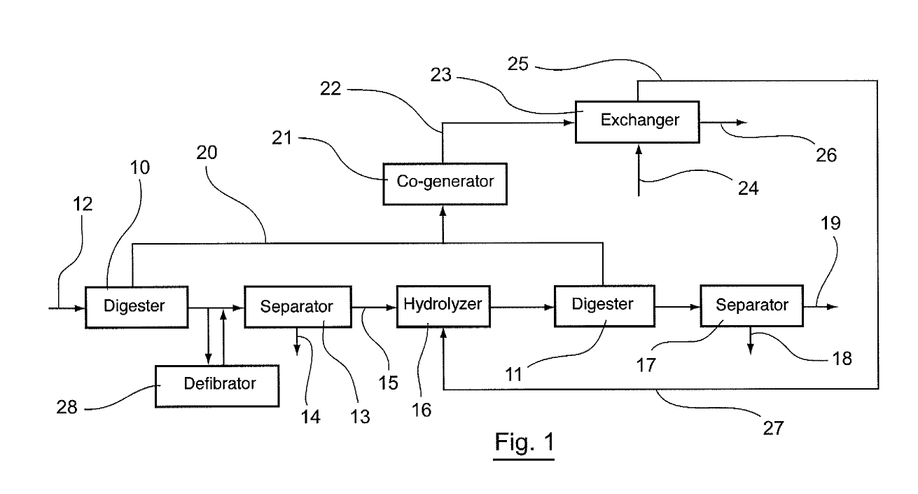

Referring to FIG. 1, we present an embodiment of a sludge treatment plant according to the invention.

As shown in this FIG. 1, such a plant comprises digestion means comprising a primary digester 10 and a secondary digester 11.

The primary digester 10 has an inlet and an outlet. The inlet is connected to sludge conveying means for leading in sludges to be treated, constituted by a piping 12. The outlet leads into the first liquid-solid separation means 13 and enables a first digestate to be poured therein.

The first liquid-solid separation means 13 include a centrifuge used to obtain a dryness greater than or equal to 12%. In one variant, any other equivalent means could be implemented for this purpose, for example membranes. These first separation means 13 have means for discharging a first effluent comprising a piping 14 and means to discharge the first dehydrated digestate comprising a piping 15. This piping 15 leads into thermal hydrolysis means 16.

The thermal hydrolysis means 16 include a reactor working under controlled conditions of pressure and temperature so as to attain the conditions for carrying out thermal hydrolysis. The thermal hydrolysis means implemented can be those described in the international patent application bearing the number WO-A1-02064516 filed on behalf of the present applicant.

The thermal hydrolysis means 16 have an outlet for discharging a hydrolyzed digestate which leads into the secondary digester 11.

The secondary digester 11 has an inlet and an outlet. The inlet is connected to the outlet of the thermal hydrolysis means 16. The outlet leads into the second liquid-solid separation means 17 and enables the hydrolyzed digestate to be poured therein.

The second separation means 17 are advantageously similar to the first separation means 13. They have means for discharging a second effluent comprising a piping 18 and means for discharging dehydrated digestate comprising a piping 19.

In one variant, these second separation means could be replaced by means for the treatment of sludges, for example by wet oxidation.

In other variants, the first and second separation means could be constituted by belt filters, filtering membranes, electro-osmotic means etc without being necessarily identical.

The primary digester 10 and the secondary digester 11 are linked to biogas recovery means. These biogas recovery means include a collector 20. The collector 20 is connected to steam and electricity producing means.

The steam production means include a co-generation motor 21. This motor is linked to an alternator which is capable of driving it in order to produce electricity.

This motor has an exhaust line 22 which leads into the inlet of an air-water heat exchanger 23.

The heat exchanger 23 has two inlets:
one inlet through which the heat produced by the co-generator 21 arrives through the piping 22;
one inlet that a water pipe 24 leads into.

It also has two outlets:
one outlet 25 for the discharge of steam;
one outlet 26 for the discharge of fumes.

The steam discharge outlet 25 is connected through a piping 27 to the thermal hydrolysis means 16.

In one variant, this installation comprises defibration means 28 positioned between the primary digester 10 and the first liquid-solid separation means 13. These defibration means 28 include a mechanical crusher. In one variant, the defibration mans 28 may include any other equivalent means for mechanically degrading (.e. removing the non-biodegradable fibrous fraction from) the first digestate coming from the first digester 10. Defibration means known to those skilled in the art are described in the international patent application number US2007/0051677. In another variant, the defibration means 28 could be positioned upstream to the primary digester.

In one variant, an exchanger will be provided between the hydrolysis means 16 and the secondary digester 11 so as to cool the sludge which exits from the hydrolysis means in order to attain the temperature conditions necessary for the secondary digestion.

### 7.3. Example of a Second Embodiment of an Installation According to the Invention

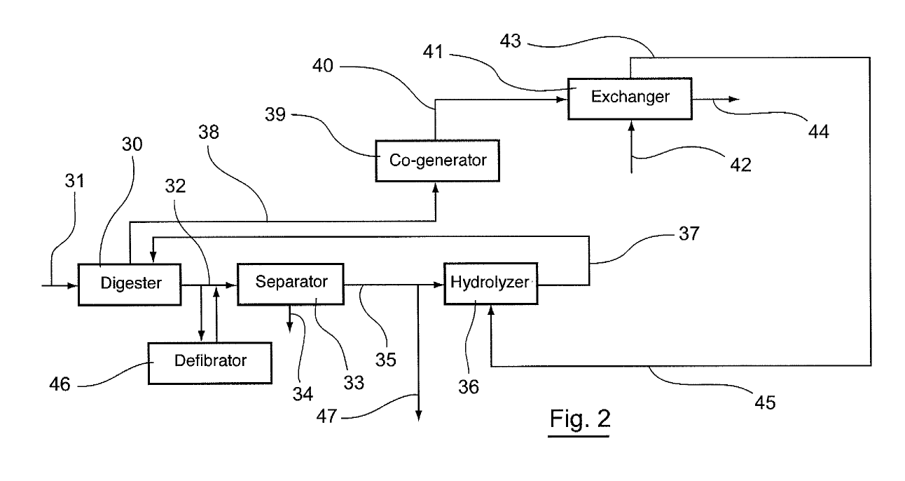

Referring to FIG. 2, we present a second embodiment of a sludge treatment plant according to the invention.

As shown in FIG. 2, such a plant includes a single digester 30. This digester 30 has a first inlet which is connected to a piping 31 for bringing in sludge to be treated. It also has an outlet for discharging a digestate that is connected to a piping 32. The piping 32 leads into the liquid-solid separation means 33.

The liquid solid separation means 33 have a structure identical to that of the liquid-solid separation means implemented in the first embodiment. These separation means 33 have means for discharging an effluent which include a piping 34 and means for discharging a dehydrated digestate which include a piping 35. This piping 35 leads into thermal hydrolysis means 36.

The thermal hydrolysis means 36 are similar to the hydrolysis means implemented in the first embodiment. They have an outlet for discharging hydrolyzed digestate which is connected by a piping 37 to a second inlet of the digester 30.

The digester 30 is connected to biogas recovery means. These biogas recovery means include a piping 38. This piping 38 is connected to means for producing steam and electricity.

The piping 35 communicates with a treated sludge discharge piping 47.

The steam production means include a co-generation motor 39. This motor is connected to an alternator which is capable of driving the motor in order to produce electricity.

This motor has an exhaust line 40 which leads into the inlet of an air-water heat exchanger 41.

The heat exchanger 41 has two inlets:
one inlet through which the heat produced by the co-generator 39 arrives through the piping 40;
an inlet that a water pipe 42 leads into. It also has two outlets:
an outlet 43 for discharging steam;
an outlet 44 for discharging fumes.

The steam discharge outlet 43 is connected through a piping 45 to the thermal hydrolysis means 36.

In one variant, the plant according to this second embodiment includes defibration means 46 which are positioned between the digester 30 and the liquid-solid separation means 33. These defibration means 46 include a mechanical crusher or any other equivalent means for mechanically degrading the digestate. In other variant, they could be placed upstream to the digester.

In one variant, an exchanger is provided between the hydrolysis means 36 and the digester 30 so as to cool the sludges which exit from the hydrolysis means in order to attain the conditions of temperature necessary for the secondary digestion. It is thus possible to recover hot water by cooling the sludges.

### 7.4. Example of a First Embodiment of a Process According to the Invention

Referring to FIG. 1, a first embodiment is presented of a process for treating sludges according to the invention.

In this process, sludges to be treated are conveyed in a primary digester 10 so that they undergo a step of primary digestion. In this embodiment, the duration of this digestion is about 10 days. In alternative embodiments, it could range from 5 to 15 days.

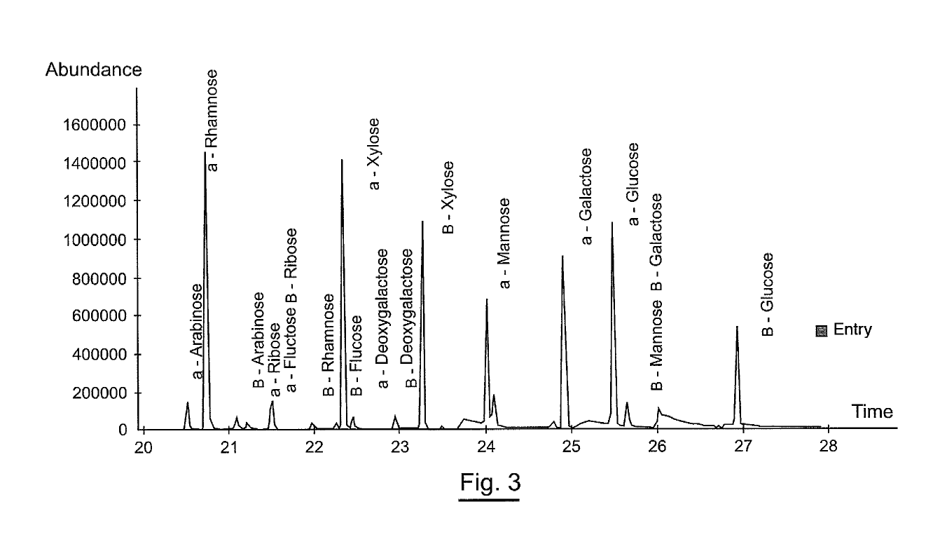

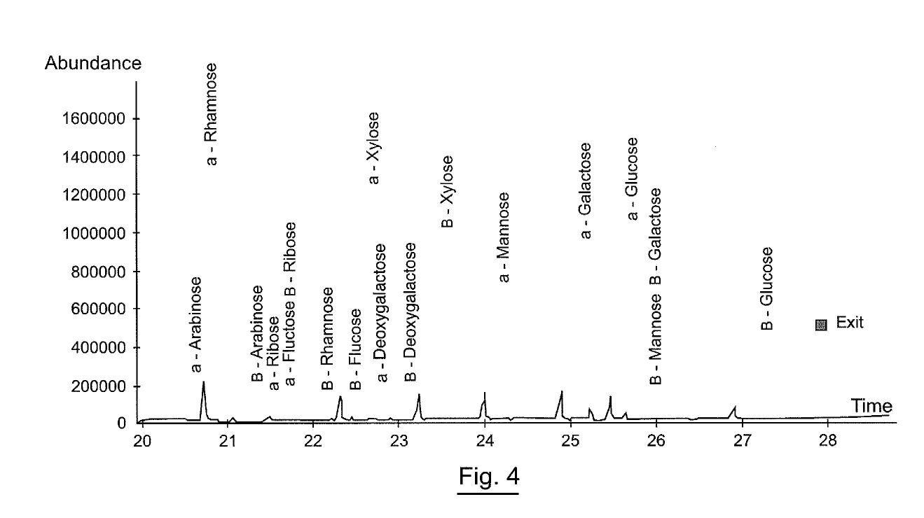

During this digestion, there is:
a reduction of the fermentable fraction of the sludges and therefore a reduction of the dry matter to be treated;
a biological hydrolysis of a part of the non-fermentable minerals (such as nitrogen and phosphorous);
an elimination of a large quantity of sugars contained in the sludges (this aspect can clearly be seen in FIGS. 3 and 4 which illustrate the sugar content of the sludges respectively before and after the implementation of the first digestion);
the generation of low bio-degradable or non-biodegradable soluble organic matter such as high COD matter and refractory nitrogen;
the solubilization of volatile fatty acids.

At the end of this digestion process, the fermentable fraction of the sludges has been digested so that the first digestate discharged at exit from the primary digester 10 is essentially constituted by the non-fermentable fraction of the sludges.

This first digestate is conveyed to the first liquid-solid separation means 13. The activation of the separation means enables the implementation of a liquid-solid separation step which leads to the production of the following:
a first effluent which flows through the piping 14;
a first dehydrated digestate having a dryness of over 12%.

The dryness of the sludge corresponds to its dry matter content calculated by deducting the percentage of humidity of the sludge from 100%.

The first effluent is rich in low-biodegradable or non-biodegradable soluble compounds formed during the primary digestion. These compounds may be:
minerals coming from the solubilization of nitrogen or phosphorous;
compounds created by organic compounds such as high COD compounds and organic nitrogen (indeed, in a classic form of digestion, between 20 and 50% of the nitrogen entering the digester exits from it in the form of NH₃) ;
compounds containing volatile fatty acids formed during the primary digestion.

Given the dryness attained during the liquid-solid separation, the dehydrated digestate is more concentrated so that its subsequent treatment requires the implementation of smaller-sized equipment and gives rise to a lower consumption of energy. All this tends to reduce the cost of treatment of the sludges.

The first dehydrated digestate is conveyed into the thermal hydrolysis means 16 in order to be subjected therein to a step of thermal hydrolysis using steam. The thermal hydrolysis is done at a temperature of 165° C., under saturation vapour pressure, for 30 minutes. In other embodiments, the hydrolysis will be done at pressure of 1 to 20 bars, a temperature ranging from 120° C. to 180° C., for 20 to 120 minutes.

Since the first dehydrated digestate comprises essentially the non-fermentable fraction of the sludge, the fermentable fraction having been digested preliminarily within the primary digester 10, the volume of the hydrolysis means is reduced by about 20 to 50% and most often by about 40% as compared with that of the hydrolysis means implemented in the prior art technique.

Furthermore, only the non-fermentable part undergoes the thermal hydrolysis treatment. The result of this is that the quantity of energy needed to make it is also substantially reduced.

Furthermore, given the fact that the liquid-solid separation undergone by the first digestate enables the discharge into the first effluent of the low biodegradable or non-biodegradable products biologically solubilized during the primary digestion, the quantity of these products that is treated during the thermal hydrolysis is reduced.

The reduction of the quantity of sugars in the hydrolyzed sludges by means of the first digestion step reduces the production of Maillard compounds, contributing to the production of hard COD material in the thermal hydrolysis step. Indeed, the Maillard reaction brings into play reduction sugars and proteins at a temperature of over 120° C. involving the formation inter alia of unreadily biodegradable soluble compounds.

Thus, although thermal hydrolysis leads to the production of low-biodegradable or non-biodegradable soluble organic compounds, these compounds are produced in relatively small quantities. The successful implementation of a primary digestion, separation and thermal hydrolysis therefore leads to the production of a smaller quantity of low-biodegradable or non-biodegradable soluble organic compounds than that produced during the successive implementation of thermal hydrolysis and digestion according to the prior art technique.

The first dehydrated digestate, made fermentable by the thermal hydrolysis treatment, is conveyed to the secondary digester 11 in order to undergo a second digestive step for 10 days. In variants, this duration could vary from 7 to 15 days.

The low-biodegradable or non-biodegradable soluble compounds produced during the primary digestion tend to disfavor against the second digestion. Thus, the preliminary elimination of these products, which limits the quantity of low-biodegradable or non-biodegradable soluble compounds produced during the hydrolysis, makes it possible to further increase the efficiency of the first digestion.

The second digestion leads to the production of a second digestate which, at least in major part, is free of the fermentable fraction and contains a unreadily biodegradable refractory part as well as a small quantity of low-biodegradable or non-biodegradable soluble organic compounds.

This mixture is conveyed to the second separation means in order to undergo a step of liquid-solid separation 17 so as to produce:
a second effluent which flows through the piping 18;
a second dehydrated digestate.

The second digestate, which is free of any fermentable fraction, at least in major part, can be re-utilized.

The digested sludges constituted by this second digestate can for example be dehydrated and then discharged or sent to another treatment step such as a wet oxidation step.

The processes of thermal hydrolysis have been implemented to improve the dehydratability of the sludges by thermal pre-treatment. The thermal hydrolysis of the digestate coming from the first digestion step also improves the dehydratability of the sludge. The implementation of an additional digestion improves the dehydratability of the digested sludges relatively to that of the raw sludges by 1 to 2%. Thus, the level of dehydration that can be attained:
on raw sludges varies from 19% to 25%;
on digested sludges varies from 21% to 30%;
on hydrolyzed sludges varies from 29% to 40%.

The second effluent is rich in low-biodegradable or non-biodegradable soluble organic compounds produced during the secondary digestion.

The first and second effluents can also be revalued or recyled at the starting point of a water treatment plant whose implementation leads to the production of sludges treated by the process according to the invention. Since the unreadily biodegradable soluble compounds are produced during the implementation of the process in small quantities as compared with the prior art technique, this recycling has a limited impact on the treated water produced.

The application of the first and second digestion steps is accompanied by the production of biogases. A recovery step enables the collection of these biogases in order to subject them to a conversion step in order to produce the steam needed to carry out the hydrolysis step and produce electricity. To this end, the biogases are conveyed into the co-generation motor 21. The application of this motor drives the alternator to which it is connected so as to produce electricity. The exhaust gases from this motor are conveyed to the exchanger 23 within which water circulates in order to produce steam. The steam thus produced is conveyed to the thermal hydrolysis means 16 through the piping 17 so as to enable the performance of the step of thermal hydrolysis of the first dehydrated digestate.

The fumes produced in the exchanger 23 are discharged through the piping 26.

### 7.5. Example of a Second Embodiment of the Process According to the Invention

Referring to FIG. 2, a second embodiment of a sludge-treatment process according to the invention is presented.

In this process, sludges to be treated are conveyed into a digester 30 so that they undergo a step of primary digestion for about 10 days. In alternative embodiments, the step could range from 5 to 15 days.

During this primary digestion, there is:
a reduction of the fermentable fraction of the sludges and therefore a reduction of the dry matter to be treated;
a biological hydrolysis of a part of the non-fermentable minerals (such as nitrogen and phosphorous);
an elimination of a large quantity of sugars contained in the sludges;
the generation of low bio-degradable or non-biodegradable soluble organic matter such as high COD matter and refractory nitrogen;
the solubilization of volatile fatty acids.

At the end of this digestion process, the fermentable fraction of the sludges has been digested so that the digestate discharged at exit from the digester 30 is essentially constituted by the non-fermentable fraction of the sludges.

This digestate is then conveyed to the separation means 33 in order to be subjected to a liquid-solid separation step. The implementation of these separation means enables the production of:
an effluent which flows through the piping 34;
a dehydrated digestate.

The effluent is rich in low-biodegradable or non-biodegradable soluble organic compounds produced during the primary digestion. These compounds may be:
minerals coming from the solubilization of nitrogen or phosphorous;
compounds created by organic compounds such as high COD compounds or organic nitrogen (indeed, in classic digestion, between 20% and 50% of the nitrogen entering the digester exits from it in the form of NH₃);
compounds containing volatile fatty acids formed during the primary digestion.

Given the dryness attained during the liquid-solid separation, the dehydrated digestate is more concentrated so that its subsequent treatment requires the implementation of smaller-sized equipment and gives rise to lower energy consumption. All this tends to reduce the cost of treatment of the sludges.

The dehydrated digestate is conveyed into the thermal hydrolysis means 36 in order to be subjected therein to a step of thermal hydrolysis under steam. The thermal hydrolysis is performed at a temperature of 165° C., at saturation vapour pressure for 30 minutes. In alternative embodiments, the hydrolysis will be done at a pressure ranging from 1 to 20 bars, a temperature of 120° C. to 180° C., for 20 to 120 minutes.

Since the dehydrated digestate comprises essentially the non-fermentable fraction of the sludges, the fermentable fraction having been preliminarily digested within the digester 30, the volume of the hydrolysis means is reduced by about 20% to 50% and most often by about 40% as compared with that of the hydrolysis means implemented in the prior art technique.

Furthermore, only the non-fermentable part of the initial sludge undergoes thermal hydrolysis treatment. The result of this is that the quantity of energy needed to carry out this treatment is also substantially reduced.

Furthermore, since the liquid-solid separation undergone by the digestate enables the discharge into the effluent of the low-biodegradable or non-biodegradable soluble compounds formed during the primary digestion, the quantity of these products that is treated during the thermal hydrolysis is reduced.

The reduction of the quantity of sugar in the hydrolyzed sludge through the first digestion step diminishes the production of Maillard compounds contributing to the production of hard COD content in the thermal hydrolysis step. Indeed, the Maillard reaction brings into play reduction sugars and proteins at a temperature of over 120° C. involving the formation, inter alia, of unreadily biodegradable soluble compounds.

Thus, although the thermal hydrolysis leads to the production of low-biodegradable or non-biodegradable soluble organic compounds, these compounds are produced in relatively small quantities. The successive implementation of primary digestion, separation and thermal hydrolysis therefore leads to the production of a smaller quantity of low-biodegradable or non-biodegradable soluble organic compounds than that produced during the successive implementation of thermal hydrolysis and digestion according to the prior art technique.

The dehydrated digestate, made fermentable by the thermal hydrolysis treatment, is recirculated in the digester 30 in which it is mixed with fresh sludge in order to make it undergo another step of digestion.

The digestion that then takes place is in fact the combination of a first digestion of fresh sludge and a second digestion of preliminarily digested and hydrolyzed sludges, this combination reducing the fermentable part of the mixture of sludges and digested sludges and leading to the production of a mix of digestates that is free, at least in major part, of fermentable fraction and contains a refractory unreadily fermentable part as well as a small quantity of low-biodegradable or non-biodegradable soluble organic compounds.

It must be noted that the portion of digestate introduced into the hydrolysis means is 100%. In other words, the entire digestate obtained at the outlet of the digester undergoes the hydrolysis treatment. In variants, the rate of recirculation of the digestate in the hydrolysis means could vary between 30% and 300%.

This mix of digestates is conveyed to the separation means 33 in order to make them undergo a step of liquid-solid separation so as to produce, as described further above:
an effluent which flows through the piping 34;
a dehydrated digestate.

The method is accomplished by setting up at least one loop, i.e. carrying out a digestion of preliminarily digested and hydrolyzed sludges.

A portion of the digestate obtained after treatment, i.e. after the setting up of at least one loop, is discharged through the piping 47 in order to be re-utilized.

This digestate may be for example dehydrated and then discharged or sent to another treatment step such as a wet oxidation step.

The methods of thermal hydrolysis have been implemented to improve the dehydratability of the sludges by thermal pretreatment. The thermal hydrolysis of the digestate coming from the first digestion step also improves the dehydratability of the sludges. The implementation of an additional digestion improves the dehydratability of the digested sludges by 1% to 2% as compared with that of raw sludges. Thus, the level of dehydration that can be attained:
on raw sludges varies from 19% to 25%;
on digested sludges varies from 21% to 30%;
on hydrolyzed sludges varies from 29% to 40%.

The collected effluent is rich in low-biodegradable or non-biodegradable soluble organic compounds produced during the secondary digestion. It can also be revalued or recirculated to the head of a water treatment plant whose implementation leads to the production of sludges which are treated by the method according to the invention. Since the unreadily biodegradable soluble compounds are produced during the implementation of the method in small quantities as compared with the prior art technique, this recycling has a reduced impact on the treated water produced.

The implementation of the first and second digestion steps is accompanied by the production of biogases. A step of recovery enables these biogases to be collected in order to subject them to a conversion step with the aim of producing the steam needed to perform the hydrolysis step and of producing electricity. To this end, the biogases are conveyed into the co-generation motor 39. The application of this motor drives the alternator to which it is connected so as to produce electricity. The exhaust gases from this motor are conveyed into the exchanger 41 within which water circulates in order to produce steam. The steam thus produced is conveyed to the thermal hydrolysis means 36 through the piping 45 so as to enable the performance of the step of thermal hydrolysis of the first dehydrated digestate.

The fumes produced in the exchanger 41 are discharged through the piping 44.

### 7.6. Other Characteristics

The operations of digestion implemented in the technique of the invention are operations of anaerobic digestion. Depending on the characteristics of the sludge to be treated, the operations of anaerobic digestion could be mesophilic or thermophilic. The temperature at which a mesophilic digestion is performed ranges from 32 to 38° C. The temperature at which a thermophilic digestion is performed ranges from 52 to 58° C. The concentration at entry of a first digester advantageously ranges from 25 grams to 65 grams of matter in suspension (MIS) per liter of sludge. The concentration at entry of a second digester advantageously ranges from 100 grams to 150 grams of matter in suspension (MIS) per liter of sludge. Should both these operations of digestion be implemented in different digesters, the characteristics of each of these digestion operations can be different. In variants, it can be planned that one or more of the operations of digestion implemented will be of the aerobic type. In variants, the digestions implemented could be of the aerobic type.

In variants, the methods of the invention described here above may include a step for subjecting the sludge, before its first entry into a digester (the first or the sole digester) or the first digestate, to a step of defibration by using the defibrator 28 or 46.

The sludge comprises a fibrous fraction that is very unreadily biodegradable in conditions of classic anaerobic digestion. At exit from the digester, this fraction may range from 30% to 60% of the organic matter present in the digestate. This fraction is almost not attacked by thermal hydrolysis. The implementation of the defibration makes it possible especially to reduce the viscosity of the sludges which advantageously have a dryness of more than 30% after defibration.

The defibration therefore makes it possible to:
treat sludge which those skilled in the art considered to be impossible with the prior art technique;
reduce the size of the digester placed upstream or downstream,
or increase the residence time of the other organic fractions of the sludge (indeed, the for an identical digester size, defibration makes it possible to reduce the fibrous fraction and therefore the quantity of dry matter entering the digester, which increases the residence time therein).

In one variant of the first and second embodiments, the first liquid-solid separation could be implemented between the thermal hydrolysis and the second digestion.

### 7.7. Energy Gains

In one technique of the prior art, the biogas produced during the digestion that follows the thermal hydrolysis is used as follows:
at least 50% of the biogas produced is fed into a boiler in order to produce the steam necessary for hydrolysis;
the remaining biogas is fed into a co-generation motor which is associated with an alternator so as to produce electricity liable to be used for a purpose other than that of the implementation of the method.

The heat of the exhaust gases from the co-generation motor can be recovered in order to produce a part of the steam needed for the thermal hydrolysis. This reduces the share of biogas used to produce steam by the implementation of a classic boiler to about 35 to 40%.

The heat released by the co-generation motor can also be recovered to pre-heat the water needed to produce steam. This reduces the share of the biogas used to produce this steam by implementing a classic boiler to 30 to 35%.

Thus, an optimal implementation of the prior art technique enables the use of 65% to 70% of the biogas produced by the digestion to produce energy likely to be used for purposes other than that of the implementation of the sludge treatment method.

According to the invention, the digestate coming from the primary digestion contains only 60% to 80% of the dry matter contained in the initial sludge. Furthermore, the digested sludges have a viscosity lower than that of raw sludge for equal dry matter content. This makes it easier to increase the dryness of the digestate obtained after the first liquid-solid separation step. The result of this is that the quantity of sludges treated by thermal hydrolysis according to the invention is appreciably smaller than that treated by thermal hydrolysis according to the prior art. Since the thermal requirements for hydrolysis are proportional to the quantity of dry matter to be hydrolyzed, the implementation of the invention reduces these thermal requirements by 30% to 40%.

Furthermore, the application of the invention increases the quantity of biogas formed during the two digestion operations by up to 20% depending on the type of sludges taken in and their residence time in the digesters.

Furthermore, the temperature of the digestate feeding the hydrolysis means is equal to about 35° C. or 55° C. depending on whether the digestion process from which it is derived is mesophilic or thermophilic.

Ultimately, the application of the invention reduces the requirements in steam needed for thermal hydrolysis by about 40% to 55% as compared with the technique of the prior art. This requirement may therefore be entirely covered by the steam obtained from the heat recovered from the exhaust gases of the motor of the co-generator. Thus, almost all the biogas produced during the digestion operations can enable the production of electrical energy that can be used for purposes other than that of the simple application of the sludge treatment method. A small quantity of the biogas produced can however be used to produce steam for starting the treatment.

However, if the requirements in steam is not entirely covered in this way in the first embodiment, then:
the digestate fed into the hydrolysis reactor could be heated by being mixed, at exit from the separation means, with hot water produced from the heat recovered either from the hydrolyzed sludges at exit from the hydrolysis reactor or from the cooling liquid and the oils of the motor of the co-generator, or from both;
the sludges feeding the first digester could be heated by being mixed with hot water produced from the recovery of heat from the hydrolyzed sludges at exit from the hydrolysis reactor.

Furthermore, the sludges fed into the second digester could be mixed with water in order to obtain optimum dryness in order to improve the performance of the second digestion operation.

In the prior art, the MIS concentration of sludge at entry into the digester is limited to 100 to 130 g/l. Indeed, the nitrogen present in the sludges gets converted into NH₃ during the digestion, NH₃ being an inhibiting compound for the digestion. It is therefore necessary to restrict the MIS concentration of the sludges at entry into the digester so as to optimize the digestion. The first digestion according to the invention substantially reduces the quantity of nitrogen contained in the sludge. Since the thermal hydrolysis of the sludges tends to reduce their viscosity the MIS concentration of the sludges at entry into the secondary digester can be increased to values ranging from 110 to 160 g/l. These sludges could therefore be mixed with water in order to attain a similar concentration of MIS.

If however the steam requirement is not entirely covered in this way in the second embodiment, then:
the digestate fed into the hydrolysis reactor could be heated by being mixed, at the outlet from the separation means, with hot water produced from the recovery of heat either from the hydrolyzed sludge at the outlet from the hydrolysis reactor or from the cooling liquid and the oils of the motor of the co-generator, or from both elements;
the sludges fed into the first digester could be heated by being mixed with hot water produced from the recovery of the heat, either on the hydrolyzed sludges at exit from the hydrolysis reactor or on the cooling liquid and the oils of the motor of the co-generator or from both.

1.-13. (canceled)

14. Method for producing essentially non-putrescible sludge and energy, said method comprising the following steps:
(i) obtaining digested sludges by primary sludge digestion;
(ii) obtaining a first aqueous effluent and digested sludges at least partly dehydrated by a first liquid-solid separation of the digested sludges obtained at the step (i);
(iii) obtaining digested sludges at least partially dehydrated and hydrolyzed by thermal hydrolysis at a temperature of 120° C. to 180° C. of the at least partially dehydrated, digested sludges obtained at the step (ii);
(iv) digestion of the at least partially dehydrated and hydrolyzed sludges obtained at the step (iii);
said process further comprising:
a step for recovering biogases formed during said digestion and said primary digestion and
a step for producing energy from said biogas comprising a sub-step for producing energy needed to implement said thermal hydrolysis and a sub-step for producing surplus energy,
the entirety of said biogas being used to produce electricity.

15. Method according to claim 14, characterized in that it comprises a step for obtaining a second aqueous effluent and treated sludges by a second liquid-solid separation of the sludges obtained at said step (iv).

16. Method according to claim 14, characterized in that said thermal hydrolysis is performed at a pressure of 1 to 20 bars, for a duration of 20 to 120 minutes.

17. Method according to claim 16, characterized in that said thermal hydrolysis is carried out at a pressure equal to the saturation vapour pressure, at a temperature of 165° C., for a duration of 30 minutes.

18. Method according to claim 14, characterized in that said primary digestion and/or said digestion are of a mesophilic anaerobic type.

19. Method according to any one of the claims 14, characterized in that said primary digestion and/or said digestion are of a thermophilic anaerobic type.

20. Method according to any one of the claims 14, characterized in that said primary digestion is preceded by a step for defibrating said sludges.

21. A system for digesting and hydrolyzing sludge, comprising:
a first digester;
a sludge influent line for directing sludge into the first digester wherein the first digester digests the sludge to form digested sludge;
a first separator disposed downstream of the first digester;
a first conduit operatively connected between the first digester and the first separator for transferring digested sludge or hydrolyzed digested sludge from the first digester to the first separator, wherein the first separator separates the digested sludge or hydrolyzed digested sludge into an effluent and a concentrated digested sludge;
a hydrolyzer disposed downstream of the first separator;
a second conduit operatively connected between the first separator and the hydrolyzer for transferring concentrated digested sludge to the hydrolyzer, wherein the hydrolyzer hydrolyzes the concentrated digested sludge to form the hydrolyzed digested sludge;
a second digester disposed downstream of the hydrolyzer or a recycle line operatively interconnected between the hydrolyzer and the first digester, and wherein the hydrolyzed digested sludge is directed to the second digester or recycled back to the first digester for further digestion;
wherein the first digester or the second digester produces biogas in the course of digesting the sludge or the hydrolyzed digested sludge; and
a collector for collecting the biogas from the first digester or the second digester, wherein the system utilizes the collected biogas to power the hydrolyzer.

22. The system of claim 21, wherein the first separator is configured to attain a dryness level equal to or greater than 12%.

23. The system of claim 21, further including a defibrator disposed generally between the first digester and the first separator.

24. The system of claim 23, wherein the defribrator receives digested sludge from the first digester and breaks down the digested sludge into smaller particles.

25. A method of treating sludge comprising the steps of:
digesting sludge to form digested sludge;
directing the digested sludge to a first separator and separating the digested sludge into an effluent and a concentrated digested sludge;
directing the concentrated digested sludge to a hydrolyzer and hydrolyzing the concentrated digested sludge to form hydrolyzed digested sludge;
further digesting the hydrolyzed digested sludge to form a second digested sludge;
directing the second digested sludge to the first separator or to a second separator and separating the second digested sludge into an effluent and a second concentrated digested sludge; and
generating biogas in the course of digesting the sludge or digesting the hydrolyzed digestive sludge and using the biogas to power the hydrolyzer.

26. The method of claim 25, wherein digestion occurs in at least two separate digestors, one disposed upstream of the hydrolyzer and one disposed downstream of the hydrolyzer.

27. The method of claim 25 wherein digestion occurs in a single digestor such that hydrolyzed digested sludge is directed back to the single digester where the hydrolyzed digested sludge is digested.

28. The method of claim 25, further including thermally hydrolyzing the digested sludge at a pressure of 1 to 20 bars, for a duration of 20 to 120 minutes.

29. The method of claim 25, further including thermally hydrolyzing the digested sludge at a pressure equal to the saturation vapour pressure, at a temperature of 165° C., for a duration of 30 minutes.

30. The method of claim 25, in which digesting the sludge or hydrolyzed digestive sludge involves a mesophilic anaerobic type digestion.

31. The method of claim 25, in which digesting the sludge or hydrolyzed digestive sludge involves a thermophilic anaerobic type digestion.

32. The method of 25, further including defibrating the sludge prior to digesting the sludge.
